(19) United States
(12) Patent Application Publication
(10) Pub. No.: US 2010/0080872 A1
(43) Pub. Date: Apr. 1, 2010

(54) LOW ACRYLAMIDE FRENCH FRY AND PREPARATION PROCESS

(75) Inventor: Susan F. Collinge, Boise, ID (US)

Correspondence Address: KELLY LOWRY & KELLEY, LLP, 6320 CANOGA AVENUE, SUITE 1650, WOODLAND HILLS, CA, 91367, US

(73) Assignee: J. R. SIMPLOT COMPANY, Boise, ID (US)

(21) Appl. No.: 12/568,424

(22) Filed: Sep. 28, 2009

Related U.S. Application Data
(60) Provisional application No. 61/101,906, filed on Oct. 1, 2008.

(51) Int. Cl. A23L 1/217; A23L 1/27; G01N 33/02; A23L 1/01

(52) U.S. Cl. 426/102; 426/250; 426/233

(57) ABSTRACT

A low acrylamide French fry potato strip and related preparation process, wherein relatively low sugar-containing potatoes are cut into pieces such as elongated French fry strips, blanched in hot water or steam, and then dipped into an aqueous solution having at least one selected food grade color agent such as annatto. The thus-dipped potato pieces are then parfried in hot oil, following by freezing for storage and/or shipment. The parfried and frozen potato pieces are adapted for finish preparation by frying in hot oil to produce French fry potato pieces such as French fry strips having a set of desirable taste, texture and appearance characteristics, in combination with a relatively low acrylamide level.

### BACKGROUND OF THE INVENTION

This invention relates generally to improved French fry potato pieces such as elongated French fry strips which, when finish prepared by frying in hot oil, exhibit a highly desirable set of taste, texture and appearance characteristics in combination with a highly desirable low acrylamide level.

Parfried and frozen potato pieces, commonly referred to as French fries, are widely available in the foods industry. These potato products are conventionally prepared by cutting whole potatoes into pieces such as elongated strips of a desired size and shape, and then partially cooking the potato strips by blanching in hot water or steam. Thereafter, the potato strips are typically dipped into an aqueous-based color development solution including dextrose. The thus-dipped potato strips are then partially fried, or parfried, in hot cooking oil, followed by freezing for packaging, shipping and/or storage. Prior to consumption, the parfried and frozen potato strips are reconstituted or finish prepared typically by finish frying in hot oil. French fried potato strips of this type are utilized extensively in restaurant and food service operations, and particularly in so-called fast food restaurants wherein it is desirable to produce a finish cooked product with a substantially optimized set of quality characteristics and with a relatively short finish fry preparation time.

More specifically, one major objective of potato processors is to provide parfried and frozen potato strips which can be finish prepared with a combination of taste, color, odor, and textural attributes selected for substantially optimum consumer palatibility. For example, it is highly desirable to provide parfried and frozen potato strips which, after finish preparation, exhibit a light and tender but crispy exterior surface of golden brown fried color encasing a soft and mealy interior which is neither too dry nor too soggy. Moreover, especially in a fast food restaurant environment, it is important to provide finish cooked potato strips which can consistently retain these desired sensory qualities for an extended holding period of at least several minutes before actual consumption. In the past, achieving these desirable product qualities on a consistent basis has generally required that the potato strips be finish prepared by frying in hot oil. In a fast food establishment, to avoid advance preparation of potato strips that might not be sold or served within a limited holding time of several minutes, a relatively short finish fry step is desired and is typically on the order of about 1.5-3.25 minutes for smaller so-called shoestring size cut strips and about 3-4.5 minutes for larger strips cut sizes.

In prior art French fry production processes, color development has been related to sugar content prior to the parfry or finish fry steps. In this regard, raw potatoes are known to exhibit an inherent or natural sugar content which increases over time as the potatoes are held in storage prior to processing. The inherent or natural sugar content of raw potatoes is periodically evaluated by sampling tests comprising test-frying of raw potato strips and then comparing the fried test strips against U.S. Department of Agriculture (USDA) color standards using a USDA-approved Munsell color chart with grades 0 through 4 available from X-Rite, Incorporated of Grandville, Mich. This natural sugar content is then normally supplemented by adding an appropriate quantity of a reducing sugar such as dextrose to an aqueous-based solution applied to the cut surfaces of production strips after blanching, and prior to parfrying. Then, when subjected to subsequent parfrying and finish frying steps, the reducing sugar cooperates with the inherent or natural raw potato sugars to insure sufficient color development so that the finish prepared products exhibit the desirable crispy exterior surface texture having the desired golden brown fried color.

While the presence of sufficient reducing sugars such as natural sugars and/or dextrose insures that the finish fried potato pieces exhibit the desirable golden brown fried color, it has been known that these reducing sugars result in proportionally increased levels of acrylamide in the finished product. In this regard, acrylamide levels on the order of 300 parts-per-billion (ppb) and higher are typical in potato pieces having a substantially optimized color development in a finish fried state. It is believed that acrylamide is produced when a reducing sugar together with the amino acid asparagine are subjected to hot oil at a relatively high parfry or finish fry temperature. Some government agencies have recently initiated attempts to reduce acrylamide in fried food products. However, attempts to produce a French fried potato product in the absence of a reducing sugar have resulted in finish fried products having insufficient color development for consumer acceptance.

There exists, therefore, a significant need for improvements in and to French fry potato processing technology, to produce a finish fried potato product having sufficient color development for consumer acceptance, while at the same time exhibiting a desirably low acrylamide level. The present invention fulfills these needs and provides further related advantages.

### SUMMARY OF THE INVENTION

In accordance with the invention, a low acrylamide French fry potato strip and related preparation process are provided, wherein cut potato pieces such as elongated French fry strips are finish fried to exhibit a desirable set of taste, texture and appearance characteristics, in combination with a desirably low acrylamide level. In a preferred form, the acrylamide level of the finish fried potato piece is less than about 100 parts-per-billion (ppb), and more preferably less than about 50 ppb.

The invention utilizes raw potatoes which are identified as having a relatively low inherent or natural sugar content. Such low sugar potatoes may comprise any of a broad range of potato varieties, including but not limited to Russet Burbank, Shepody, and others, recognizing that the inherent or natural sugar content of the raw potatoes is generally low at the time of harvesting and tends to increase over time as the potatoes are stored awaiting processing. In one preferred form, low sugar potatoes are identified by initial samples testing wherein selected potatoes are cut into approximate shoestring-size elongated strips, and a central-region strip is selected from each sample potato for frying in hot oil. The thus-fried sample strips are then compared with U.S. Department of Agriculture (USDA) color standards using a USDA-approved Munsell color chart with grades 0 through 4 available from X-Rite, Incorporated of Grandville, Mich. More particularly, low sugar potatoes are defined as those fried sample strips having a color grade of 1 or less, and more preferably a color grade of 0.

The identified low sugar potatoes are production processed by optional peeling, following by cutting into pieces such as elongated shoestring-size French fry strips, and then blanched in hot water or steam. The blanched potato pieces are then dipped into or otherwise surface-coated with an aqueous solution having at least one selected food grade color agent such as annatto. In a preferred embodiment, the dip solution includes annatto at about 0.05 weight percent. Alternative and/or additional dip solution color agents such as turmeric, beta carotene and the like may also be used. This dip solution does not include any substantial addition of a reducing sugar such as dextrose.

The thus-dipped potato pieces are then parfried in hot oil, following by freezing for storage, packaging, and/or shipment. The parfried and frozen potato pieces are adapted for finish preparation by frying in hot oil to produce French fry potato pieces such as French fry strips having a set of desirable taste, texture and appearance characteristics, in combination with a relatively low acrylamide level. In the preferred form, the finish fried pieces have color development of from about 50 to about 80 on the Agtron colorimeter scale, and more preferably about 55 to about 70 on the Agtron scale. In addition, the finish fried pieces have an acrylamide level of less than about 100 ppb, and more preferably less than about 50 ppb.

Other features and advantages of the invention will become more apparent from the following detailed description.

### DETAILED DESCRIPTION OF THE PREFERRED EMBODIMENTS

The present invention relates to an improved French fry potato pieces and related preparation process, wherein finish fried elongated potato strips exhibit a desirable combination of taste, appearance, and texture characteristics, together with a relatively low acrylamide level.

In general terms, the potato pieces are cut from raw potatoes having a natural or inherent relatively low sugar content, and these cut potato pieces such as elongated strips are processed substantially in the absence of any added reducing sugar. Specifically, raw potatoes having a relatively low natural sugar content are identified, cut into pieces such as French fry strips, blanched in hot water or steam, and then subjected to an aqueous solution as by dipping or spraying wherein the aqueous solution includes at least one food grade color agent such as annatto. Thereafter, the potato pieces are parfried in hot oil, and frozen for shipping and/or packaging to await finish fry preparation. The frozen potato pieces are finish fried in hot oil for consumption. In the finish fried state, the potato pieces exhibit a highly desirable set of taste and texture characteristics in combination with perceived color development corresponding closely with conventionally prepared (i.e., with reducing sugar, such as a higher natural sugar content and/or dextrose applied in a conventional pre-parfry dip solution) French fry potato strips, but wherein the finish prepared potato pieces further exhibit a dramatically reduced acrylamide level. In a preferred form, this acrylamide level is less than about 100 parts-per-billion (ppb), and more preferably, less than about 50 ppb.

More specifically, in accordance with the invention, raw whole potatoes of a type and variety exhibiting a natural relatively low sugar content are identified and selected for use in the production process and resultant product of the present invention. More particularly, raw potatoes are selected by lot (i.e., common variety and substantially common harvest date). Selected potatoes from the lot are initially sample-tested to identify relatively low sugar potatoes for further processing in accordance with the invention. More particularly, these selected potatoes are initially cut as by hand-cutting a selected number of the potatoes (such as 20 potatoes) into smaller pieces such as elongated shoestring-size French fry strips. Of these cut strips, a single centrally located strip is selected from each sample potato for subsequent frying in hot oil for about 3 minutes at a temperature of about 375 F. The thus-fried sample strips are then compared against U.S. Department of Agriculture (USDA) color standards using a USDA-approved Munsell color chart with grades 0 through 4 available from X-Rite, Incorporated of Grandville, Michigan. Relatively low sugar potatoes are defined as those potatoes exhibiting a color grade of 1 or less, and more preferably a color grade of 0. Exemplary low sugar potatoes may comprise, e.g., newly harvested white potatoes of a broad variety range, including but not limited to Russet Burbank, Shepody, and others.

The production lot of the identified relatively low sugar raw potatoes are preferably although optionally peeled, and then cut into potato pieces having a desired size and shape, such as elongated shoestring-size potato strips having a generally square cross-sectional shape with cut sides having a dimension of about 0.30 inch, and a length distribution ranging from about 2 inches to about 6 inches. These raw-cut potato pieces are initially blanched in hot water or steam, as by blanching in hot water at about 165 F. for about 8 minutes.

The blanched potato pieces are then subjected to a surface application of an aqueous solution, as by dipping or spraying, wherein the aqueous solution includes at least one food grade color agent but specifically excludes any reducing sugar such as dextrose. In particular, in accordance with one preferred form, the aqueous solution comprises a dip solution having about 0.3% weight sodium acid pyrophosphate (SAPP), and about 0.05% weight annatto, at a temperature of about 150 F. The pH of the dip solution is adjusted to about 7.0. The blanched potato pieces are immersed within this dip solution for about 30 seconds.

Additional agents can be added to the aqueous solution, such as acrylamide inhibitors including, for example, an amino acid such as lysine and/or a pH adjustment solution such as monosodium citrate.

The thus-dipped potato pieces are removed from the dip solution and dried for up to about 10 minutes in warm air at a temperature of about 140 F., and a relative humidity of about 20%. This dry step achieves a weight loss for the potato pieces of about 10-15%.

The dipped and dried potato pieces are then parfried in hot oil at a temperature within a range of from about 360 to about 400 F., and preferably within a range of from about 375 to about 395 F., for a time period of about 45 seconds. The parfried pieces are removed from the hot parfry oil and then frozen. In the parfried and frozen state, the potato pieces exhibit a moisture content of from about 62% to about 68% by weight.

For finish preparation, the parfried and frozen potato pieces are finish fried in hot oil at a conventional time and temperature range, typically from about 330 to about 370 F., with a preferred finish fry temperature being toward the lower end of this temperature range for further reducing potential acrylamide formation. In the finish fried state, the potato pieces exhibit a highly desirable set of taste and texture characteristics closely matching conventionally prepared (i.e., with color development attributable to the presence of natural and/or reducing sugar such as dextrose in a conventional pre-parfry dip solution) French fry potato strips. In addition, the finish fried potato pieces also exhibit a highly desirable light golden brown color development which also closely matches the color of conventionally pre-processed and finish fried potato strips, namely, a finish fry color reading of from about 50 to about 80, and more preferably about 55 to about 70, of the Agtron colorimeter scale. However, the finish fried prepared potato pieces further exhibit a dramatically reduced acrylamide level. In a preferred form, this acrylamide level is less than about 100 ppb, and, more preferably, less than about 50 ppb.

An alternative colorimeter scale is the CIELAB (Commission Internationale de l'Eclairage L*a*b) scale using colorimeter Model No. Chroma Meter CR-310, marketed by Konica Minolta Corporation Sensing Americas, Inc., of Ramsey, N.J. With this colorimeter, preferred finish fries exhibit a color reading with an “L” range (light to dark) of from about 55 to about 85, with a more preferable reading range of about 65 to about 75; an “a” range reading (green to red) of between about −5 to about +5, and more preferably between about −3 to about +3; and a “b” range reading (blue to yellow) of between about +15 to about +35, and more preferably between about +19 to about +30. See http:dba.med.sc.edu/price/irf/Adobe tg/models/cielab.html for a more detailed explanation of the CIELAB color comparison scale, which is incorporated by reference herein.

The following examples illustrate the invention:

### EXAMPLE 1

A “control” sample was prepared comprising conventional shoestring size potato strips as described above cut from typical peeled Russet Burbank raw potatoes. These raw potatoes were peeled and cut into elongated shoestring size potato strips, dipped after blanching (as described above) into an aqueous solution containing about 0.3% weight sodium acid pyrophosphate (SAPP), and about 0.3% weight reducing sugar (dextrose)—an appropriate level of reducing sugar to achieve a desired color development when the strips are finish fried. These dipped strips were dried, parfried, and frozen as previously described herein. Upon finish fry preparation, the finished potato strips comprising this “control” sample exhibited a combination of desirable taste, texture, and appearance (including color development) characteristics, together with a conventional acrylamide level of about 323 ppb.

### EXAMPLE 2

Relatively low sugar raw potatoes identified as variety AOA9515A (having a sample test color grade reading of about 0 on the USDA-approved Munsell color chart) were similarly peeled and cut into shoestring size elongated potato strips as described generally in Example 1, except that these cut strips were subjected after blanching to a modified aqueous dip solution containing about 0.3% weight SAPP, and about 0.05% weight annatto (as described above), in the absence of added reducing sugar such as dextrose. The thus-dipped potato pieces were then dried, parfried, and frozen as described above to await finish fry preparation. After finish frying, the potato pieces exhibited taste, color and texture characteristics closely matching the “control” of Example 1, but wherein the finish prepared potato pieces had a significantly reduced acrylamide level of about 22 ppb.

### EXAMPLE 3

Alternative relatively low sugar potatoes identified as Premier Russets (having a sample test color grade reading of about 0 on the USDA-approved Munsell color chart) were processed according to Example 2. After finish frying, the potato pieces exhibited taste, color and texture characteristics closely matching the “control” of Example 1, but wherein the finish prepared potato pieces had a significantly reduced acrylamide level of about 36 ppb.

### EXAMPLE 4

Alternative relatively low sugar Russet Burbank potatoes (having a sample test color grade reading of about 0 on the USDA-approved Munsell color chart) were processed according to Example 2. After finish frying, the potato pieces exhibited taste, color and texture characteristics closely matching the “control” of Example 1, but wherein the finish prepared potato pieces had a reduced acrylamide level of about 75 ppb.

### EXAMPLE 5

Alternative relatively low sugar Umatilla variety potatoes (having a sample test color grade reading of about 0 on the USDA-approved Munsell color chart) were processed according to Example 2. After finish frying, the potato pieces exhibited taste, color and texture characteristics closely matching the “control” of Example 1, but wherein the finish prepared potato pieces had a reduced acrylamide level of about 81 ppb.

### EXAMPLE 6

Conventional Russet Burbank potatoes having a traditional natural sugar content, i.e., a sample test color grade reading of about 2-3 on the USDA-approved Munsell color chart, were cut and processed according to Example 2. That is, the conventional sugar potatoes were dipped into the aqueous dip solution which contained about 0.3% weight sodium acid pyrophosphate (SAPP), and about 0.05% weight annatto (as described above), in the absence of added reducing sugar such as dextrose, but in the presence of a comparatively higher natural sugar content (in comparison with Examples 2-5). The thus-dipped potato pieces were then dried, parfried, and frozen as described above to await finish fry preparation. After finish frying, the potato pieces exhibited taste, color and texture characteristics closely matching the “control” of Example 1, but wherein the finish prepared potato pieces had a substantially higher acrylamide level of about 179 ppb.

Accordingly, the combination of identifying and using relatively low sugar potatoes (defined as having a sample test color grade reading of 1 or less, and more preferably about 0, on the USDA-approved Munsell color chart), with a post-blanch, pre-parfry dip solution containing a food grade color agent such as annatto but excluding any added reducing sugar such as dextrose, produces finish fried potato pieces exhibiting the desirable taste, appearance, and texture characteristics with the added benefit of a significantly lower acrylamide level. Persons skilled in the art will recognize and appreciate that this dip solution may include batter coating constituents such as flours, starches, dextrin, hydrocolloids, salt and the like, but excluding reducing sugars.

A variety of alternative food grade color agents and/or combinations thereof will be apparent to persons skilled in the art. For example, such alternative and/or additional food grade color agents may include or comprise annatto, turmeric, beta-carotene, and others, and mixtures thereof. Accordingly, no limitation on the invention is intended by way of the foregoing description, except as set forth in the appended claims.

What is claimed is:

1. A process for producing a parfried French fry potato strip for subsequent finish preparation with a relatively low acrylamide level, said process comprising the steps of:
selecting raw potatoes having a relatively low natural sugar content;
cutting the raw potatoes into elongated strips;
blanching the elongated potato strips;
applying an aqueous solution including at least one food grade coloring agent to coat external surfaces of the blanched potato strips; and
parfrying the coated potato strips;
whereby the parfried potato strips, when finish prepared, exhibit an acrylamide level of less than about 100 ppb.

2. The process of claim 1 wherein the parfried potato strips, when finish prepared, exhibit an acrylamide level of less than about 50 ppb.

3. The process of clam 1 further comprising the step of freezing the parfried potato strip prior to finish preparation.

4. The process of claim 1 further comprising the step of finish preparing the parfried potato strips by finish frying in hot oil to exhibit a color within the range of from about 50 to about 80 on the Agtron colorimeter scale.

5. The process of claim 4 wherein the finish fried potato strips exhibit a color with the range of from about 55 to about 70 on the Agtron colorimeter scale.

6. The process of claim 1 wherein said selecting step comprises the steps of cutting at least one centrally located strip from a lot of selected potatoes, finish frying the centrally located strips from said lot of selected potatoes, comparing the finish fried strips against a USDA-approved Munsell color chart having color grades 0 through 4, and selecting potatoes exhibiting a color grade of 1 or less.

7. The process of claim 6 wherein said selecting step comprises the step of selecting potatoes exhibiting a fried color grade of about 0.

8. The process of claim 1 wherein said applying step is selected from the group consisting of dipping and spraying.

9. The process of claim 1 wherein the aqueous solution includes annatto.

10. The process of claim 1 wherein the aqueous solution includes said food grade coloring agent substantially in the absence of reducing sugar.

11. The process of claim 10 wherein the aqueous solution includes said food grade coloring agent comprising annatto at about 0.05% by weight.

12. The process of claim 11 wherein said aqueous solution further comprises about 0.3% weight sodium acid pyrophosphate, and has a temperature of about 150° F. and a pH of about 7.0, said applying step comprising immersing the blanched potato pieces in said aqueous for about 30 seconds.

13. The process of claim 1 further comprising the step of partially drying the potato strips subsequent to said aqueous solution applying step and prior to said parfrying step, said partially drying step achieving a potato strip weight loss of from about 10% to about 15%.

14. The process of claim 1 wherein said parfrying step comprises parfrying the potato strips in hot oil at a time and temperature to achieve a parfried potato strip moisture content within the range of from about 62% to about 68% by weight.

15. A process for producing a parfried French fry potato strip for subsequent finish preparation with a relatively low acrylamide level, said process comprising the steps of:
selecting raw potatoes having a relatively low natural sugar content, said selecting step comprising the steps of cutting at least one centrally located strip from a lot of selected potatoes, finish frying the centrally located strips from said lot of selected potatoes, comparing the finish fried strips against a USDA-approved Munsell color chart having color grades 0 through 4, and selecting potatoes exhibiting a color grade of 1 or less;
cutting the selected raw potatoes into elongated strips;
blanching the elongated potato strips;
applying an aqueous solution including at least one food grade coloring agent substantially in the absence of a reducing sugar to coat external surfaces of the blanched potato strips;
parfrying the coated potato strips to a parfried strip moisture content within the range of from about 62% to about 68% by weight; and
finish preparing the parfried potato strips to exhibit a color within the range of from about 50 to about 80 on the Agtron colorimeter scale, whereby the finish prepared potato strips exhibit an acrylamide level of less than about 100 ppb.

16. The process of claim 15 wherein the finish prepared potato strips exhibit an acrylamide level of less than about 50 ppb.

17. The process of claim 15 wherein said selecting step comprises selecting potatoes having a color grade of about 0.

18. The process of claim 15 wherein finish preparing step comprises finish frying the potato strips in hot oil to exhibit a color within the range of from about 55 to about 70 on the Agtron colorimeter scale.

19. The process of claim 15 wherein the aqueous solution includes annatto.

20. The product prepared in accordance with the process of claim 15.
